(19) United States
(12) Plant Patent Application Publication
(10) Pub. No.: US 2003/0188359 P1
(43) Pub. Date: Oct. 2, 2003

(54) Ornamental sweetpotato plant named 'Sweet Caroline Light Green'

(76) Inventors: Kenneth Pecota, Raleigh, NC (US); G. Craig Yencho, Washington, NC (US); Cynthia Pierce, Clayton, NC (US)

Correspondence Address: MYERS BIGEL SIBLEY & SAJOVEC, PO BOX 37428, RALEIGH, NC, 27627, US

(21) Appl. No.: 10/114,386

(22) Filed: Apr. 2, 2002

(51) Int. Cl. A01H 5/00

(52) U.S. Cl. PLT/258

(57) ABSTRACT

A new and distinct ornamental cultivar of Ipomoea batatas called ‘Sweet Caroline Light Green’ is described that is a moderately-compact plant with light green leaves that are moderately- to deeply-lobed. The plant is distinguished by its many short shoots, dense foliage, chartreuse leaves, and purple band at the leaf base. Furthermore, this plant has excellent vigor and flowers under short day conditions. Ipomoea batatas ‘Sweet Caroline Light Green’ is suitable for use in landscaping and containerized gardens.

### LATIN NAME OF THE GENUS AND SPECIES

The Latin name of the novel, ornamental plant variety disclosed herein is Ipomoea batatas (L.) Lam.

### VARIETY DENOMINATION

The inventive cultivar of Ipomoea batatas disclosed herein has been given the variety denomination ‘Sweet Caroline Light Green’.

### BACKGROUND OF THE INVENTION

Ipomoea species are members of the morning glory family Convolvulaceae. Ipomoea batatas, commonly referred to as the white or yellow sweetpotato and the orange yam, are typically fast growing vines with palmately-lobed leaves. These ornamental species produce storage roots identical in appearance to the common sweet potato, but not as palatable. Late in the growing season, tubular flowers appear which are similar to morning glories, but plantings are dominated by the appearance of the foliage. The plants are highly desirable due to their ability to grow under varied stress conditions, cover a large space, and last the entire growing season. Moreover, these plants have few insect or disease problems.

Existing varieties of Ipomoea batatas are popular for landscaping applications. There are currently six common types of ornamental sweetpotatoes that are being cultivated primarily for annual, summer vines. These six cultivars are: ‘Blackie’, having dark purple-black foliage, lavender flowers, and edible storage roots; ‘Terrace Lime’ and ‘Margarita’ (also known as ‘Sulfur’), which have large brilliant chartreuse leaves and lavender blooms; ‘Black Heart’ (also known as ‘Ace of Spades’), having heart-shaped leaves with burgundy purple color; ‘Tricolor’ (also known as ‘Pink Frost’), is a variegated plant which has pale green, white, and pink-margined leaves; and ‘Lady Fingers’, which has medium green, dainty leaves divided into long, thin, fingerlike lobes which are complemented by burgundy stems and veins.

Ipomoea batatas ‘Margarita’ has recently been released in the United States, and has become widely used as a landscape annual. It is not suitable for mixed containers, as this variety exhibits a very vigorous growth and tends to out-compete other species. Another popular variety is ‘Blackie’, a vigorous purple-leaved clone which is also unsuited to containerized gardens. See Armitage. A. M. and J. M. Garner. 2001. Ipomoea batatas ‘Margarita’. HortScience 36:178.

Therefore, to meet the current horticultural demand, it is desirable to produce new, more robust cultivars of ornamental sweetpotato with attractive foliage colors, leaf shapes, and plant architectures. In addition, it would be advantageous to develop cultivars of ornamental sweetpotato exhibiting a more compact growth, and which do not out-compete other species in mixed containers.

The present invention comprises a new and distinct variety of Ipomoea batatas, which has been named ‘Sweet Caroline Light Green’. The variety is suitable for use as a landscape or containerized plant.

Lineage.

The Ipomoea batatas ‘Sweet Caroline Light Green’ cultivar originated from a conventional cross between Ipomoea batatas cultivars NC15-2ORN (the female parent; not patented) and NC136-4ORN (the male parent; not patented) conducted in the Winter of 1999-2000 at the Horticultural Greenhouses located at North Carolina State University, Raleigh, N.C. NC15-2ORN resulted from a cross between ‘Sulfur’ and a ‘P5’ (origin unknown) by ‘Sulfur’ cross. NC136-4ORN resulted from a cross between ‘Sulfur’ and a ‘Sulfur’ by ‘Blackie’ cross. Seeds from this cross were planted in the Horticultural Greenhouses in Spring 2000. The single, individual plant now known as Ipomoea batatas ‘Sweet Caroline Light Green’ was selected in July 2000 because of its combination of exceptional features, and has been propagated asexually since that time.

Asexual Reproduction.

Since its selection, Ipomoea batatas ‘Sweet Caroline Light Green’ has been asexually reproduced at the Horticultural Greenhouses located at North Carolina State University, Raleigh, N.C. predominantly by vegetative propagation of vine cuttings. Successively, there have been two cycles of vegetative propagation, one cycle of tissue culture micropropagation, and multiple vegetative propagation cycles to increase the plant population. The combination of characteristics disclosed herein for Ipomoea batatas ‘Sweet Caroline Light Green’ has remained stable through the successive generations of asexual reproduction.

### BRIEF SUMMARY OF THE INVENTION

The present Ipomoea batatas ‘Sweet Caroline Light Green’ ornamental plant is a moderately-compact, densely-mounding cultivar producing many, short shoots and having dense foliage. This cultivar is distinguishable from other cultivars by its bright chartreuse green leaves that have 3-5 moderate to deep-lobes. The plant has a good vigor, but is less vigorous than Ipomoea batatas ‘Margarita’ and ‘Blackie’ and, unlike these cultivars, ‘Sweet Caroline Light Green’ may be grown in containers with other species. ‘Sweet Caroline Light Green’ flowers under short day conditions.

### BRIEF DESCRIPTION OF THE DRAWINGS

FIG. 1 is a color photograph of a typical specimen of the Ipomoea batatas ‘Sweet Caroline Light Green’ grown in a container under commercial greenhouse conditions.

FIG. 2 shows the variety of leaves produced by Ipomoea batatas ‘Sweet Caroline Light Green’ and the lower surface of the leaf (bottom row, center leaf).

FIGS. 3A to 3C shows several views of typical flowers produced by Ipomoea batatas ‘Sweet Caroline Light Green.’

FIG. 3A shows a side view of a flower with a petal peeled back to reveal interior structures.

FIG. 3B shows a side view of an intact flower.

FIG. 3C is a top view of an intact flower.

FIG. 4 provides a comparison of Ipomoea batatas ‘Sweet Caroline Light Green’ (right) with the commercially available ‘Margarita’ cultivar (left).

### DETAILED BOTANICAL DESCRIPTION

The following is a detailed description of the botanical characteristics of a new and distinct cultivar of Ipomoea batatas plant known by the cultivar name ‘Sweet Caroline Light Green’. All colors cited herein refer to The Royal Horticultural Society Colour Chart designations except where general terms of ordinary dictionary significance are used. Where dimensions, sizes, colors, and other characteristics are given, it is to be understood that such characteristics are approximations or averages set forth as accurately as practicable.

The descriptions reported herein are from 12-week-old specimens grown individually in six-inch azalea pots. The plants were grown in Lompoc, Calif., under commercial practice in a polycarbonate-covered greenhouse during the 2001-2002 winter season with day and night temperatures ranging between 18.3-26.7° C. and 15.6-18.3° C., respectively, and light levels of about 4,000-8,000 foot-candles. Ipomoea batatas ‘Sweet Caroline Light Green’ has not been observed under all possible environmental conditions, therefore, the phenotype may vary under different environmental conditions such as season, temperature, light intensity, day length, cultural conditions, and the like.

Growth Conditions.

Ipomoea batatas ‘Sweet Caroline Light Green’ has excellent vigor, has a moderately fast growth rate, and is very adaptable to container culture. In locales with mild winter conditions, Ipomoea batatas ‘Sweet Caroline Light Green’ will grow perennially; otherwise it is an annual plant. Similar to cultivated sweetpotatoes, wind or rain rarely causes much damage to ‘Sweet Caroline Light Green’, but if damage does occur, the plant drops the damaged leaves and grows new shoots at nodes where the leaves were lost. Under low light levels in a greenhouse, ‘Sweet Caroline Light Green’ can develop intumescence, which will remain on the affected foliage, but will be outgrown with new foliage.

Above-Ground Structure and Coloration.

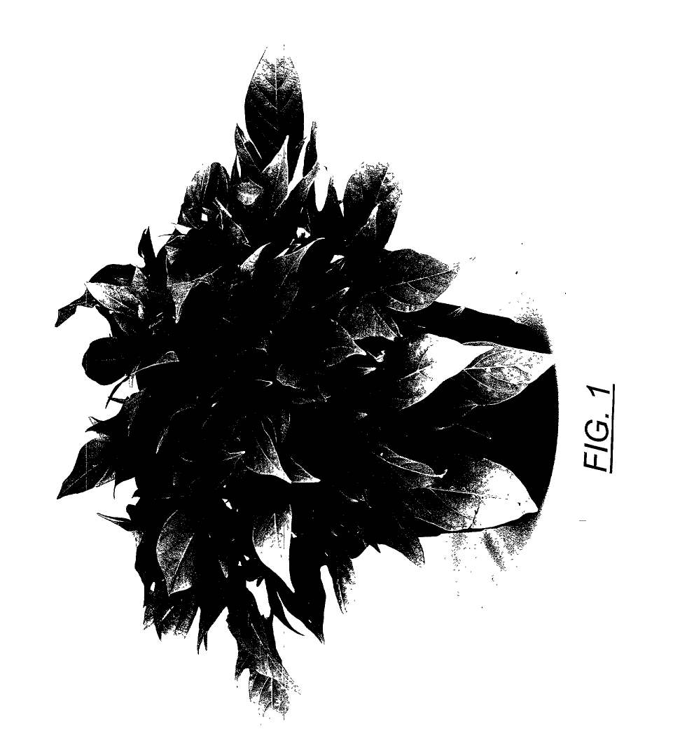

FIG. 1 shows the shape and coloration of a typical specimen of Ipomoea batatas ‘Sweet Caroline Light Green’. Overall, this cultivar is a moderately-compact, mound shaped, herbaceous plant that has an average height of 16 cm and an average area of spread of 38×40 cm. The growth habit of this plant is to grow upright with shoots growing outward.

Branching Habitat.

Freely-branching with ˜14 lateral branches coming off the stem. No basal branching. Very dense foliage with no pinching required to stimulate branching.

Vegetative Lateral Branches.

Length: ˜12 cm. Diameter: ˜0.4 cm. Internodes are short with an average length of ˜1.0 cm. Many lateral branches are formed and each axil has latent shoots.

Stem.

Round and smooth with an outward and upward bending aspect and very good strength. Color: 144C.

Petiole.

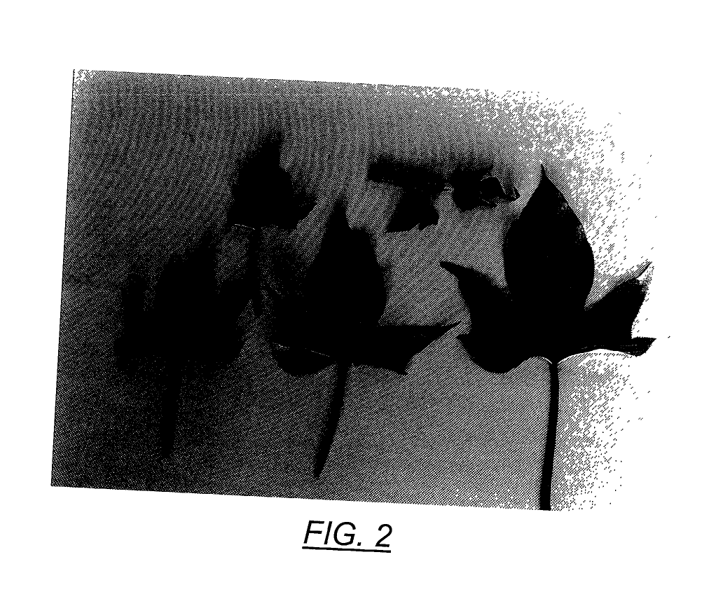

Length: ˜11.0 cm. Diameter: ˜0.3 cm. Color: 145B. A very distinct, purple (185B), 0.5 cm band is present at the leaf base at the junction of the petiole with the leaf blade (see FIG. 2)

Foliage.

Leaves are alternate and simple, but deeply divided into 3-5 lobes. Leaf shape is consistent but size varies (see FIG. 2). Quantity: Very densely foliated, with ˜12-14 leaves per lateral branch. Mature leaf length: ˜12.4 cm. Mature leaf width: ˜10.5 cm. Leaf margin is entire. Lobe width: ˜1.8 cm. Mid-vein lobe length: ˜9.5 cm. Mid-vein lobe width: ˜4.8 cm. Leaf apex: Acute. Leaf base: Acute. Leaf has a smooth texture and matte finish. Venation is palmate at the base with arcuate veins in the center lamina. Color: Table 1. Leaves start out yellow-green and become lighter as they mature.

TABLE 1

| Leaf Structure | Upper Surface | Lower Surface |
|---|---|---|
| Young Leaf | 144B | 144B |
| Mature Leaf | 151A/B | 145C |
| Vein | 151C | 145D |

Flowers.

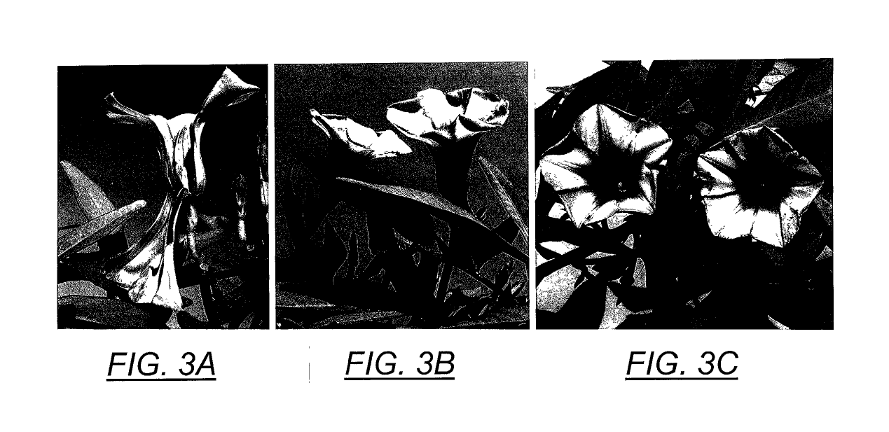

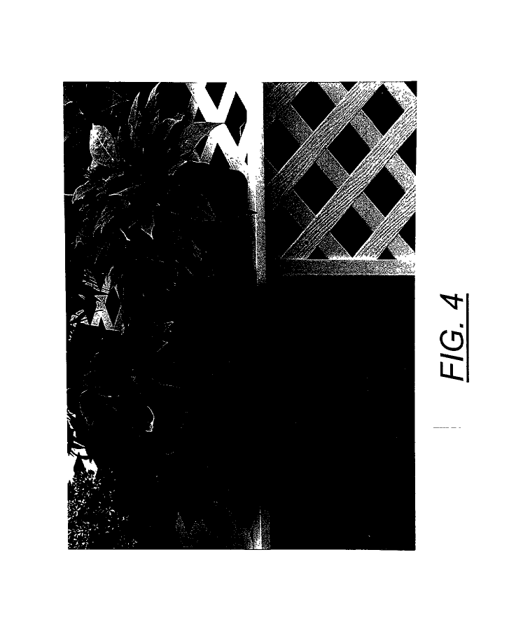

‘Sweet Caroline Light Green’ flowers under short day conditions, but the precise photoperiod for flower induction is currently unknown. FIGS. 3A, 3B and 3C show views of typical flowers of the variety. The inflorescence is generally a cyme in which the peduncle is divided into two axillary peduncles. Each peduncle is further divided into two after the flower is produced. Usually buds of the first, second and third order are developed, but sometimes single flowers are produced. Corolla width: ˜3.9 cm, corolla length: ˜4.4 cm. Limb color: 69C. The throat color gets lighter from base to limb going from 77A at the base to 77C near the limb. The limb is round or pentagonal. The two outer sepals are shorter than the inner sepals. The sepals are lanceolate and green in color. The stigma and style are both white. The stigma is exerted relative to the stamens. There is some variation in flower size and color, depending on the environmental conditions. Descriptions are based on: CIP, AVRDC, IBPGR. 1991. Descriptors for Sweet Potato. Huaman, Z., editor. International Board for Plant Genetic Resources, Rome, Italy.

Root Coloration.

The roots have a light rose skin with cream-colored flesh.

Comparison With Other Ipomoea batatas Cultivars.

Of the six most common cultivars of ornamental sweetpotato, Ipomoea batatas ‘Sweet Caroline Light Green’ most resembles the ‘Margarita’ and ‘Terrace Lime’ cultivars. Like ‘Margarita’ and ‘Terrace Lime’, Ipomoea batatas ‘Sweet Caroline Light Green’ has light green leaves. However, where ‘Margarita’ and ‘Terrace Lime’ have large, slightly-lobed leaves, ‘Sweet Caroline Light Green’ has moderately-sized and moderately- to deeply-lobed leaves. Moreover, ‘Sweet Caroline Light Green’ has a moderately-compact plant habit as compared with the trailing habit of ‘Margarita’. Furthermore, the storage root of ‘Sweet Caroline Light Green’ has a light rose skin and cream-colored flesh as compared with the light purple,skin and cream-colored flesh of ‘Margarita’.

In a comparison with the parental strains, ‘Sweet Caroline Light Green’ shares attributes with each parent (Table 2), but is also quite distinct therefrom, and comprises a unique combination of characteristics.

TABLE 2

| | ‘Sweet Caroline | Female Parent | Male Parent |
|---|---|---|---|
| Characteristic | Light Green’ | NC15-2ORN | NC136-4ORN |
| Plant Habit | Moderately Compact | Trailing | Moderately |
| | | | Compact |
| Foliage Color | Light Green | Light Green | Bronze |
| Leaf Size | Moderate | Large | Moderate |
| Leaf Shape | Moderately-to | Slightly-Lobed | Deeply-Lobed |
| | Deeply-Lobed | | |

Disease or Pest Resistance.

‘Sweet Caroline Light Green’ is susceptible to Sweetpotato Feathery Mottle Virus and damage by Japanese beetles.

Herbarium voucher.

A voucher of ‘Sweet Caroline Light Green’ was obtained by Dr. Paul R. Fantz and Dr. Robert Lyons, Department of Horticultural Science, North Carolina State University, Raleigh, N.C., on Jul. 6, 2001. The voucher number is 6575, and the voucher will eventually be deposited into the Herbarium of North Carolina State University (NCSC) in Raleigh, N.C.

What is claimed is:

1. A new and distinct cultivar of Ipomoea batatas plant named ‘Sweet Caroline Light Green’, substantially as illustrated and described herein.
